(19) United States
(12) Patent Application Publication
(10) Pub. No.: US 2013/0131724 A1
(43) Pub. Date: May 23, 2013

(54) Chiropractic Device Having Alignment-by-Touch Function

(71) Applicant: Masanori Harada, Fukuoka (JP)

(72) Inventor: Masanori Harada, Fukuoka (JP)

(21) Appl. No.: 13/811,920

(22) PCT Filed: Dec. 3, 2010
(86) PCT No.: PCT/JP2010/071643

§ 371 Date: Jan. 24, 2013

(30) Foreign Application Priority Data
Aug. 9, 2010 (JP) 2010-178647

(51) Int. Cl. A61F 5/00

(52) U.S. Cl. CPC A61F 5/00
USPC 606/237

(57) ABSTRACT

Chiropractic apparatus including a chiropractic adjuster means and a contact means. The contact means being capable of relative movement with respect to the chiropractic adjuster means, the contact means having a forward end portion that is capable of contacting both the tip of a palpation finger of a user and a body surface of a patient, the contact means having a targeting space that is capable of receiving the tip and causing the palpation fingertip to be placed in contact with the body surface. The targeting space receiving the forward end portion of the chiropractic adjuster means and causing the forward end portion to be placed in contact with the body surface. By subjecting the contact means and the chiropractic adjuster means to relative movement, the chiropractic adjuster means is capable of moving to the targeting space of the contact means and placed in contact with the body surface.

### FIELD OF THE INVENTION

The present invention relates to a chiropractic apparatus having a targeting function by tactile sensation. More particularly, the present invention is concerned with a chiropractic apparatus comprising the following means (i) and (ii): (i) a chiropractic adjuster means, and (ii) a contact means attached to the chiropractic adjuster means, the contact means being capable of relative movement with respect to the chiropractic adjuster means,

the contact means having a forward end portion which is capable of contacting both the tip of a palpation finger of a user and a body surface of a patient,

the contact means having, at the forward end portion thereof, a targeting space which is capable of receiving the tip of the palpation finger of the user and causing the palpation fingertip to be placed in contact with the body surface of the patient,

the targeting space being for receiving the forward end portion of the chiropractic adjuster means and causing the forward end portion to be placed in contact with the body surface of the patient,

wherein, by subjecting the contact means and the chiropractic adjuster means to relative movement with respect to each other, the forward end portion of the chiropractic adjuster means is capable of moving to the targeting space at the forward end portion of the contact means so as to be placed in contact with the body surface of the patient.

By the use of the chiropractic apparatus of the present invention having a targeting function by tactile sensation, it becomes possible that a target site for chiropractic treatment is determined by palpation using a fingertip that is most sensitive in a human body, and then a thrust is correctly applied to the target site. Therefore, by the use of the chiropractic apparatus of the present invention, chiropractic treatment can be performed more effectively and efficiently. Also, the chiropractic apparatus of the present invention is easily portable.

### BACKGROUND OF THE INVENTION

The term “chiropractic” means “hand action” and is a combination of Greek words “Chiro” (“hand”) and “Prakticos” (“action”). As indicated by its name, in the field of chiropractic, palpation using various parts of the hands, such as a fingertip and the pisiform bone, is performed on the body of a patient for determining a target site for chiropractic treatment, and then chiropractic treatment is performed by a method in which a thrust (i.e., an impact of high speed and low amplitude) is applied to the target site by using various parts of the hands or by using an apparatus employing spring resilience or electromagnetic force. In the field of chiropractic, amelioration of various diseases and ailments is achieved by applying an appropriate thrust to the cranium, the spine, the lumbar vertebrae, the pelvis, and/or joints of limbs so as to correct small dislocation of these bones. (In the field of chiropractic, such small dislocation of bones is called “subluxation” or “misalignment”.) When a portion, to be given a thrust, of the body surface of the patient has a skin slack, the thrust applied is disadvantageously absorbed by the skin slack, thus making it impossible to transmit a satisfactory level of force of thrust to the target site; therefore, generally, before the application of a thrust to the body surface of the patient, the skin of the portion, to be given a thrust, of the body surface of the patient is pulled so as to be placed under tension to remove any skin slack. In the field of chiropractic, the term “thrust” covers any type of mechanical pressures. Examples of methods for applying a mechanical pressure include a method in which a fingertip of a chiropractic practitioner or a forward end of a rod (made of a plastic or metal) is placed on the body surface of a patient, and then the fingertip or rod is macroscopically moved to apply a pressure (thrust) to the body surface of the patient (this is, for example, the case where a thrust is applied by using the body of a chiropractic practitioner or the case where a thrust is applied by using a so-called “activator” described below), and a method in which a forward end of a rod (made of a plastic or metal) is placed on the body surface of a patient, and then a mechanical impulse is applied to a rear end of the rod (which is not macroscopically moved) thereby generating a thrust in the form of a pressure wave propagating from the rear end to forward end of the rod to thereby apply a pressure (thrust) to the body surface of the patient (this is the case where there is used an apparatus for performing a so-called “Atlas Orthogonal Technique” described below).

In the field of chiropractic, chiropractic treatment (application of a thrust) is usually called “adjustment”. Therefore, hereinafter, application of a thrust is frequently referred to as “adjustment”. (In the field of chiropractic, the term “manipulation” may be used as having the same meaning as “adjustment”.) In the field of chiropractic, a portion, to be given a thrust, of the body surface of the patient is usually referred to as a “contact point”. Therefore, hereinafter, a portion, to be given a thrust, of the body surface of the patient is frequently referred to as a “contact point”. With respect to the general information of chiropractic and to the above-mentioned various terms, reference can be made to, for example, Non-Patent Document 1 (“Kairopurakutikku-Gairon (Outline of Chiropractic)”, authored by Seikyo SUZUKI, published by Taniguchi Shoten Publishing Co., Japan, 1987), Non-Patent Document 2 (“Hajimete Manabu Kairopurakutikku-Sukiru-Kihon-Gensoku-Kara Manipyureishon-Sukiru-Made (Introduction to Chiropractic Skills—from basic principles to manipulation skills)”, authored and edited by David Byfield, translation supervised by Motoaki OTANI, published by IDO-NO-NIPPON SHA INC., Japan, 1999), and Non-Patent Document 3 (“Okai-DC-No Tekunikku-Bukku, Kihon-Hen, Jissen-Kairopurakutikku-Ajasutomento-Tekunikku (Technique Book of Okai DC (Basics) Practical Chiropractic Adjustment Techniques)”, authored by Takeshi OKAI, published by Japan Medical Publishing Co. LTD., Japan, 2004). With respect especially to the term “adjustment”, reference is made to Non-Patent Documents 1 and 3.

When an adjustment is performed using a finger, such as an index finger or middle finger, problems may arise not only in that a burden is imposed on the finger and the wrist of the chiropractic practitioner (chiropractor), thus causing the chiropractor to suffer injuries, but also in that the direction of the thrust is not correct, thus making it impossible to obtain a desired effect on the patient. When it is necessary to apply a high force thrust, the pisiform bone is used in many cases. However, the pisiform bone is less sensitive as compared to a fingertip, and therefore, a high level skill is required to perform a correct adjustment by using the pisiform bone.

For solving these problems accompanying adjustments performed using the human power, adjustments may be performed using equipment (chiropractic apparatuses). In the case of the use of chiropractic apparatuses, a thrust is applied to the patient by using spring resilience or electromagnetic force. Conventional chiropractic apparatuses are described in, for example, U.S. Patent Application Publication No. US 2002/0082532 A1 (Patent Document 1), U.S. Pat. No. 6,379,375 (Patent Document 2), U.S. Pat. No. 6,602,211 (Patent Document 3), and U.S. Pat. No. 6,805,700 (Patent Document 4). These chiropractic apparatuses are generally referred to as “activators”, and the chiropractic techniques performed using such chiropractic apparatuses may be referred to as “Activator Methods Chiropractic Technique (AMCT)”. (Further, in the field of chiropractic, chiropractic apparatuses are frequently referred to as “adjusters”, because they are devices used for performing “adjustment”. Therefore, hereinafter, a chiropractic apparatus is frequently referred to as an “adjuster”.)

When using any of the above-mentioned conventional chiropractic apparatuses to apply a thrust, one hand of the user is occupied by holding and operating the chiropractic apparatus, so that the other hand of the user is used for touching the body of the patient. Therefore, in this case, the operation for pulling the skin of the portion of the body surface of the patient corresponding to a target site for adjustment so as to remove a skin slack, is performed using only the other hand not holding the chiropractic apparatus. Such one-hand operation for pulling the skin to remove a skin slack is not easy, as compared to the case of a two-hand operation for pulling the skin to remove a skin slack. Further, there is also the following problem. A contact point is determined by performing palpation using a fingertip of the user. Subsequently, the palpation fingertip of the user is once released from the determined contact point, and then a forward end of the chiropractic apparatus is moved toward and placed on the contact point by relying on the visual memory of the user. Due to the reliance on the visual memory, frequently, the forward end of the chiropractic apparatus is slightly misplaced from the correct contact point determined by the palpation. For these reasons, in the case of the use of the above-mentioned conventional chiropractic apparatuses, performing a correct and effective adjustment is not easy even for a skilled chiropractor.

There is known “Atlas Orthogonal Technique” (AOT), which is a subfield of chiropractic. In the Atlas Orthogonal Technique, the target of an adjustment is limited to the first cervical vertebra (the atlas), i.e., C1. In the case of the Atlas Orthogonal Technique, a large-sized, floor-mounted machine specialized for adjusting the atlas is used. In this large-sized, floor-mounted machine, a chiropractic apparatus (adjuster) is attached to a movable arm which can hold the chiropractic apparatus stably. In the case of the use of this large-sized machine, by virtue of the fact that the chiropractic apparatus can be held stably, a skilled chiropractor can perform an adjustment of the atlas relatively easily. However, this large-sized machine is specialized for adjusting the atlas and cannot be used for an adjustment of a bone other than the atlas.

A chiropractic apparatus which can solve the above-mentioned problems accompanying the prior art has not yet been developed.
[Patent Document 1] U.S. Patent Application Publication No. US 2002/0082532 A1
[Patent Document 2] U.S. Pat. No. 6,379,375
[Patent Document 3] U.S. Pat. No. 6,602,211
[Patent Document 4] U.S. Pat. No. 6,805,700
[Non-Patent Document 1] “Kairopurakutikku-Gairon (Outline of Chiropractic)”, authored by Seikyo SUZUKI, published by Taniguchi Shoten Publishing Co., Japan (1987)
[Non-Patent Document 2] “Hajimete Manabu Kairopurakutikku-Sukiru-Kihon-Gensoku-Kara Manipyureishon-Sukiru-Made- (Introduction to Chiropractic Skills—from basic principles to manipulation skills)”, authored and edited by David Byfield, translation supervised by Motoaki OTANI, published by IDO-NO-NIPPON SHA INC., Japan (1999)
[Non-Patent Document 3] “Okai-DC-No Tekunikku-Bukku, Kihon-Hen, Jissen-Kairopurakutikku-Ajasutomento-Tekunikku (Technique Book of Okai DC (Basics) Practical Chiropractic Adjustment Techniques)”, authored by Takeshi OKAI, published by Japan Medical Publishing Co. LTD., Japan (2004)

### DISCLOSURE OF THE INVENTION

### Problems to be Solved by the Invention

Thus, it has been desired to develop a chiropractic apparatus which can solve the above-mentioned problems accompanying the prior art, i.e., a chiropractic apparatus which is not only easily portable, but also enables even a chiropractor not having a high level skill to perform easily a correct and effective adjustment of any bone of the entire body skeleton of a patient.

### Means to Solve the Problems

In this situation, the present inventor has made extensive and intensive studies with a view toward solving the above-mentioned problems. As a result, he has unexpectedly found that the above-mentioned problems can be solved by a chiropractic apparatus comprising the following means (i) and (ii): (i) a chiropractic adjuster means, and (ii) a contact means attached to the chiropractic adjuster means, the contact means being capable of relative movement with respect to the chiropractic adjuster means,

the contact means having a forward end portion which is capable of contacting both the tip of a palpation finger of a user and a body surface of a patient,

the contact means having, at the forward end portion thereof, a targeting space which is capable of receiving the tip of the palpation finger of the user and causing the palpation fingertip to be placed in contact with the body surface of the patient,

the targeting space being for receiving the forward end portion of the chiropractic adjuster means and causing the forward end portion to be placed in contact with the body surface of the patient,

wherein, by subjecting the contact means and the chiropractic adjuster means to relative movement with respect to each other, the forward end portion of the chiropractic adjuster means is capable of moving to the targeting space at the forward end portion of the contact means so as to be placed in contact with the body surface of the patient.

The present invention has been completed, based on this finding.

The foregoing and other objects, features and advantages of the present invention will be apparent from the following detailed description taken in connection with the accompanying drawings, and the appended claims.

### Effects of the Invention

By the use of the chiropractic apparatus of the present invention having a targeting function by tactile sensation, it becomes possible that a target site for chiropractic treatment (adjustment) is determined by palpation using a fingertip that is most sensitive in a human body, and then a thrust is correctly applied to the target site. Therefore, by the use of the chiropractic apparatus of the present invention, an adjustment can be performed more effectively and efficiently. Also, the chiropractic apparatus of the present invention is advantageous not only in that it is easily portable, but also in that an adjustment can be performed even in a state where both hands of a user are kept in contact with the body of a patient, thereby enabling even a chiropractor not having a high level skill to perform easily a correct and effective adjustment of any bone of the entire body skeleton of a patient.

### BRIEF DESCRIPTION OF THE DRAWINGS

FIG. 1 is a diagrammatic front view of an example of the chiropractic apparatus.

FIG. 2 is a diagrammatic perspective view of another example of the chiropractic apparatus.

FIG. 3 is a diagrammatic perspective view of still another example of the chiropractic apparatus.

FIG. 4 is a diagrammatic perspective view of still another example of the chiropractic apparatus.

FIG. 5 is a diagrammatic cross-sectional view of still another example of the chiropractic apparatus.

FIG. 6 is a diagrammatic front view showing how to operate the chiropractic apparatus.

FIG. 7 is a diagrammatic view showing the internal structure of an example of a chiropractic adjuster means.

FIG. 8 is a diagrammatic view showing the internal structure of another example of a chiropractic adjuster means.

### DESCRIPTION OF REFERENCE NUMERALS

1 Chiropractic apparatus
2 Chiropractic adjuster means
2A Forward end portion (thrust head) of a chiropractic adjuster means
2B Trigger member
2C Coil compression spring
2D Thrust driving block
2E Thrust rod
2F Coil compression spring
2G Coil compression spring
3 Contact means
3A Forward end portion of a contact means
3B Shaft
3C Spring holding member
3D Spring holding member
3E Coil spring
3F Circumferential pipe wall region of a forward end opening of a pipe which is a contact means
3G Hole in a circumferential pipe wall region of a forward end opening of a pipe which is a contact means
4 Targeting space
F Finger of a user
P Body surface of a patient

### BEST MODE FOR CARRYING OUT THE INVENTION

In the present invention, there is provided a chiropractic apparatus comprising the following means (i) and (ii):

(i) a chiropractic adjuster means comprising:
a thrust member extending in a longitudinal direction of the chiropractic adjuster means and having a thrust head constituting a forward end portion of the chiropractic adjuster means,
a driving member for applying a longitudinal forward thrust to the thrust member, and
a trigger member for operating the driving member,
   (ii) a contact means attached to the chiropractic adjuster means, the contact means being capable of relative movement with respect to the chiropractic adjuster means,
the contact means having a forward end portion which is capable of contacting both the tip of a palpation finger of a user and a body surface of a patient,
the contact means having, at the forward end portion thereof, a targeting space which is capable of receiving the tip of the palpation finger of the user and causing the palpation fingertip to be placed in contact with the body surface of the patient,
the targeting space being for receiving the forward end portion of the chiropractic adjuster means and causing the forward end portion to be placed in contact with the body surface of the patient,

wherein, by subjecting the contact means and the chiropractic adjuster means to relative movement with respect to each other, the forward end portion of the chiropractic adjuster means is capable of moving to the targeting space at the forward end portion of the contact means so as to be placed in contact with the body surface of the patient.

Next, for easier understanding of the present invention, the essential features and various preferred embodiments of the present invention are enumerated below.

1. A chiropractic apparatus comprising the following means (i) and (ii):

(i) a chiropractic adjuster means comprising:
a thrust member extending in a longitudinal direction of the chiropractic adjuster means and having a thrust head constituting a forward end portion of the chiropractic adjuster means,
a driving member for applying a longitudinal forward thrust to the thrust member, and
a trigger member for operating the driving member,
   (ii) a contact means attached to the chiropractic adjuster means, the contact means being capable of relative movement with respect to the chiropractic adjuster means,
the contact means having a forward end portion which is capable of contacting both the tip of a palpation finger of a user and a body surface of a patient,
the contact means having, at the forward end portion thereof, a targeting space which is capable of receiving the tip of the palpation finger of the user and causing the palpation fingertip to be placed in contact with the body surface of the patient,
the targeting space being for receiving the forward end portion of the chiropractic adjuster means and causing the forward end portion to be placed in contact with the body surface of the patient,

wherein, by subjecting the contact means and the chiropractic adjuster means to relative movement with respect to each other, the forward end portion of the chiropractic adjuster means is capable of moving to the targeting space at the forward end portion of the contact means so as to be placed in contact with the body surface of the patient.

2. The chiropractic apparatus according to item 1 above, wherein the forward end portion of the contact means comprises an arc, and the inside of the arc is the targeting space.

3. The chiropractic apparatus according to item 1 above, wherein the forward end portion of the contact means comprises a loop, and the inside of the loop is the targeting space.

4. The chiropractic apparatus according to item 1 above, wherein the forward end portion of the contact means comprises a plate, the plate having upper and lower surfaces and a through-hole extending between the upper and lower surfaces, wherein the through-hole is the targeting space,

wherein the chiropractic adjuster means is positioned on the side of the upper surface of the plate, and the lower surface of the plate is capable of being placed in contact with the body surface of the patient.

5. The chiropractic apparatus according to item 1 above, wherein the contact means comprises an elastic pipe having rear and forward end openings,

the rear end opening of the elastic pipe having inserted therein a forward region of the chiropractic adjuster means,

the forward end opening of the elastic pipe being the targeting space, and

wherein the forward end opening of the elastic pipe has a circumferential pipe wall region having therein a finger insertion hole which is capable of receiving the tip of the palpation finger of the user and causing the palpation fingertip to be positioned in the targeting space.

Hereinbelow, the present invention is described in detail with reference to the accompanying drawings.

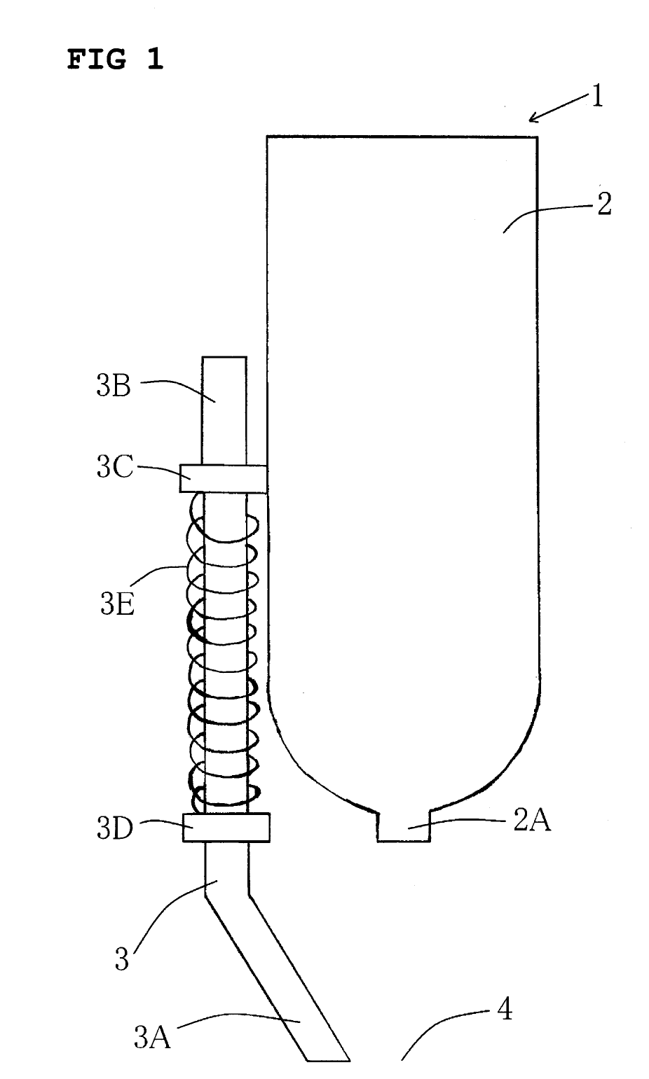

FIG. 1 shows an example of the chiropractic apparatus of the present invention. As shown in FIG. 1, chiropractic apparatus 1 of the present invention comprises chiropractic adjuster means 2 (hereinafter, “chiropractic adjuster means” is frequently referred to simply as “adjuster means”) and contact means 3.

With respect to adjuster means 2, explanation thereon is given later.

Contact means 3 shown in FIG. 1 has shaft 3B, spring-holding member 3C, spring-holding member 3D and coil spring 3E. Coil spring 3E may be either a compression spring or a tension spring. Coil spring 3E is wound around shaft 3B. Spring-holding member 3C is attached to adjuster means 2. The attachment of spring-holding member 3C to adjuster means 2 can be made by conventional methods, such as welding, fusion bonding, adhesion and screwing (e.g., attaching spring-holding member 3C to adjuster means 2 by means of a screw; and screwing spring-holding member 3C to adjuster means 2). Spring-holding member 3C holds coil spring 3E from above, and spring-holding member 3D holds coil spring 3E from below.

Spring-holding member 3C also has a through-hole, and holds shaft 3B slidably trough the through-hole. The expansion and contraction of coil spring 3E causes shaft 3B to slide through the through-hole of spring-holding member 3C, thereby enabling contact means 3 to move upward and downward. Thus, in the embodiment shown in FIG. 1, contact means 3 is capable of relative movement with respect to adjuster means 2, wherein contact means 3 can move in the longitudinal direction of adjuster means 2.

Forward end portion 3A of contact means 3 is capable of contacting both the tip of palpation finger F of the user and body surface P of the patient. Contact means 3 has targeting space 4 at the neighborhood of forward end portion 3A of contact means 3. Targeting space 4 is positioned right under forward end portion 2A of adjuster means 2. Targeting space 4 is a space which is capable of receiving the tip of palpation finger F of the user and causing the palpation fingertip to be placed in contact with body surface P of the patient and which is for receiving the forward end portion of the adjuster means and causing the forward end portion to be placed in contact with the body surface of the patient.

By moving adjuster means 2 downward while holding contact means 3 on body surface P of the patient, forward end portion 2A of adjuster means 2 is capable of moving to targeting space 4 so as to be placed in contact with the body surface of the patient.

With respect to the shape of forward end portion 3A of contact means 3, there is no particular limitation. Forward end portion 3A of contact means 3 shown in FIG. 1 has a rod shape, but it is not required that the forward end portion of the contact means have a rod shape. For example, the forward end portion of the contact means may have a curved shape, such as an arcuate shape or a quasi-arcuate shape. Alternatively, the forward end portion of the contact means may comprise a loop. Further, the forward end portion of the contact means may comprise a plate.

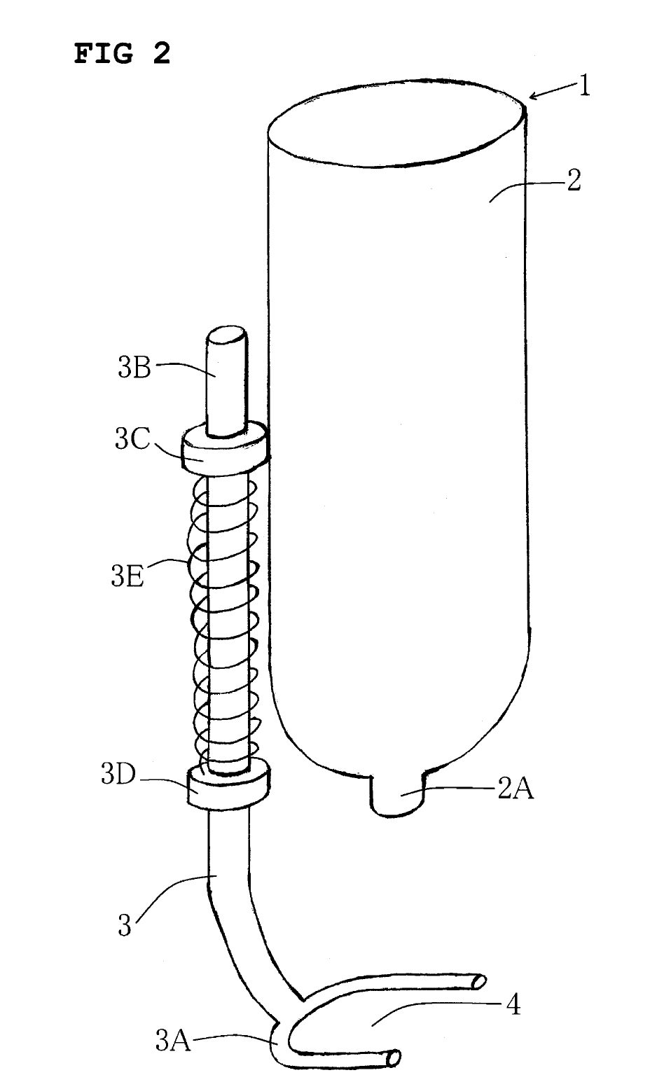

FIG. 2 shows another example of the chiropractic apparatus of the present invention. In this example, forward end portion 3A of contact means 3 comprises an arc. In FIG. 2, the inside of the forward end portion having an arcuate shape is targeting space 4.

With respect to the size of the arcuate forward end portion, the explanation, given later, of the size of a loop applies, wherein the explanation is made in connection with the case where forward end portion 3A of contact means 3 comprises the loop. (The arcuate forward end portion is regarded as a loop (circle) containing as a part thereof the arcuate forward end portion.)

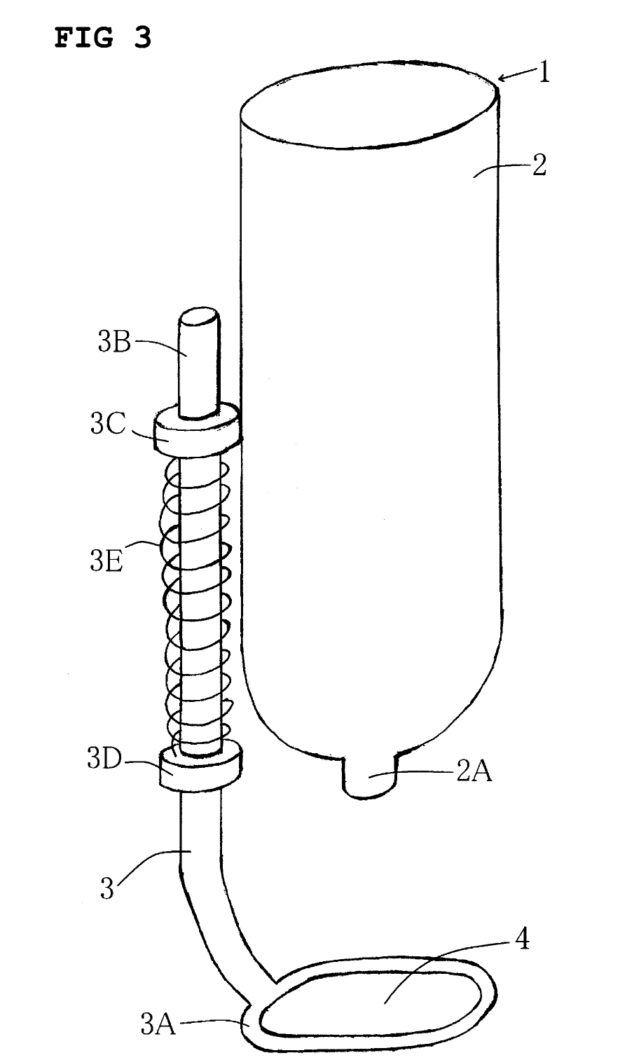

FIG. 3 shows still another example of the chiropractic apparatus of the present invention. In this example, forward end portion 3A of contact means 3 comprises a loop. In FIG. 3, the inside of the loop is targeting space 4. With respect to the shape of the loop, there is no particular limitation. The loop shown in FIG. 3 has a circular shape, but it is not required that the loop have a circular shape. As examples of shapes of the loop other than a circular shape, there can be mentioned those of ellipses and polygons. Examples of polygons include a triangle (e.g., an equilateral triangle), a tetragon (e.g., a square), a pentagon and a hexagon. Further, the loop is not required to have a complete form of loop. For example, the loop may be a discontinuous one, which does not form a closed curve.

The loop has a size such that each of the tip of palpation finger F of the user and forward end portion 2A of adjuster means 2 can pass through the inside of the loop. With respect to the size of the loop, the explanation, given later, of the size of a through-hole applies, wherein the explanation is made in connection with the case where forward end portion 3A of contact means 3 comprises a plate having the through-hole.

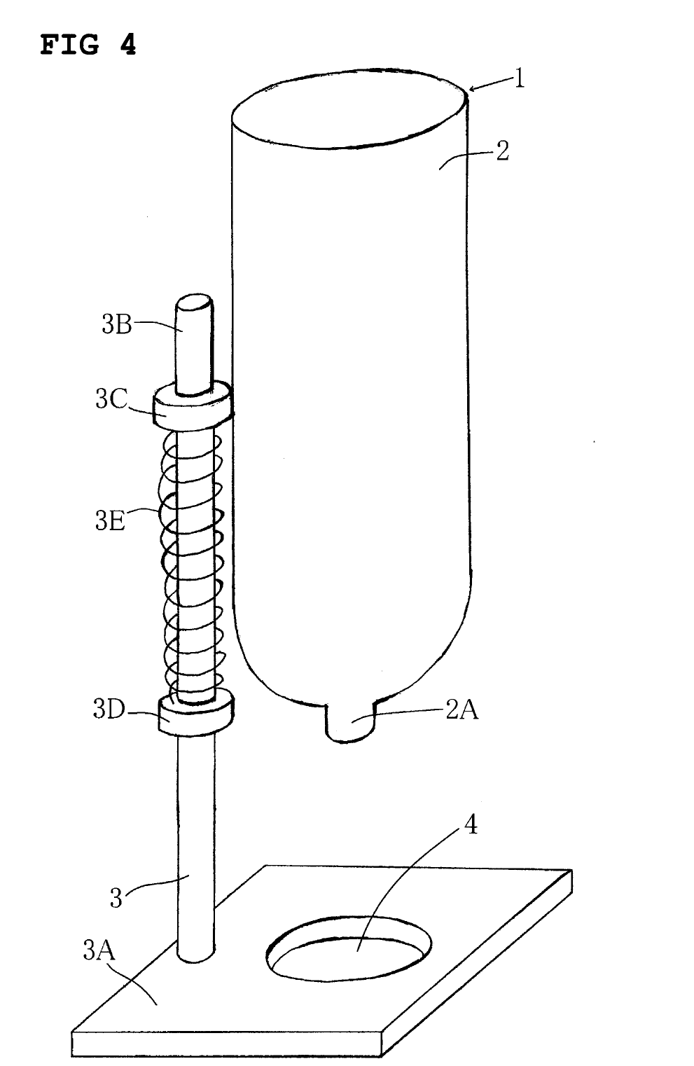

FIG. 4 shows still another example of the chiropractic apparatus of the present invention. In this example, forward end portion 3A of contact means 3 comprises a plate. Plate 3A has upper and lower surfaces and a through-hole extending between the upper and lower surfaces, wherein the through-hole is targeting space 4. Adjuster means 2 is positioned on the side of the upper surface of plate 3A, and the lower surface of plate 3A is capable of being placed in contact with body surface P of the patient.

The through-hole (targeting space 4) of plate 3A has a size such that each of the tip of palpation finger F of the user and forward end portion 2A of adjuster means 2 can pass through the through-hole. With respect to the size of the through-hole of plate 3A, there is no particular limitation so long as each of the tip of palpation finger F of the user and forward end portion 2A of adjuster means 2 can pass through the through-hole.

More specifically, the size of the through-hole of plate 3A is as follows. The through-hole of plate 3A has a size such that the diameter of the largest circle which the through-hole can contain is generally from 3 mm to 50 mm, preferably from 3 mm to 25 mm (hereinafter, “diameter of the largest circle which the through-hole can contain” is frequently referred to simply as “diameter of the through-hole”). The lower limit of the diameter of the through-hole of plate 3A is more preferably 5 mm, still more preferably 8 mm. The upper limit of the diameter of the through-hole of plate 3A is more preferably 17 mm, still more preferably 12 mm.

The tip of palpation finger F of the user is generally thicker than forward end portion 2A of adjuster means 2 and, therefore, it is desired to determine the diameter of the through-hole in accordance with the thickness of the tip of palpation finger F of the user. It is preferred that the diameter of the through-hole is the same as or slightly smaller (e.g., by about 1 mm to about 3 mm) than the maximum diameter of the tip of palpation finger F of the user. (The tip of palpation finger F of the user has flexibility to some extent and, therefore, the tip of palpation finger F of the user can pass through the through-hole even when the diameter of the through-hole is slightly smaller than the maximum diameter of the tip of palpation finger F of the user.) It is added for information that with respect to the adjusters used in the field of the Atlas Orthogonal Technique, the forward end portion of the thrust head of the adjusters generally has a diameter of about 3 mm, while with respect to the adjusters used for other purposes (i.e., portable adjusters), the forward end portion of the thrust head of the adjusters has a diameter in the range of from about 10 mm to about 15 mm. With respect to the tip of palpation finger F of the user, the maximum diameter thereof is about 25 mm or less, and is generally in the range of from abut 10 mm to about 15 mm. When the diameter of the through-hole is larger than the maximum diameter of the tip of palpation finger F of the user (in other words, in the case where a gap is formed, during the palpation, between the tip of palpation finger F of the user and the peripheral wall of the through-hole), it is preferred to perform the palpation while positioning the tip of palpation finger F at the center of the largest circle which the through-hole can contain, so that the portion, to be given a thrust, of the body surface of the patient can be determined in a state where the tip of palpation finger F is positioned at the center of the circle. After the palpation thus performed, forward end portion 2A of adjuster means 2 is moved into the through-hole of plate 3A so as to position forward end portion 2A at the center of the largest circle which the through-hole can contain. In this way, a more correct adjustment can be achieved.

With respect to the shape of the through-hole of plate 3A, there is no particular limitation. As examples of shapes of the through-hole of plate 3A, there can be mentioned those of circles, ellipses and polygons (such as a triangle (e.g., an equilateral triangle), a tetragon (e.g., a square) and a pentagon (e.g., an equilateral pentagon) and those of regions surrounded by closed curves other than mentioned above. Plate 3A may have a break in a part of the periphery of the through-hole. For example, there may be a crack or cut (slit) extending from the through-hole to an end of plate 3A, so that the through-hole is communicated with the end of plate 3A.

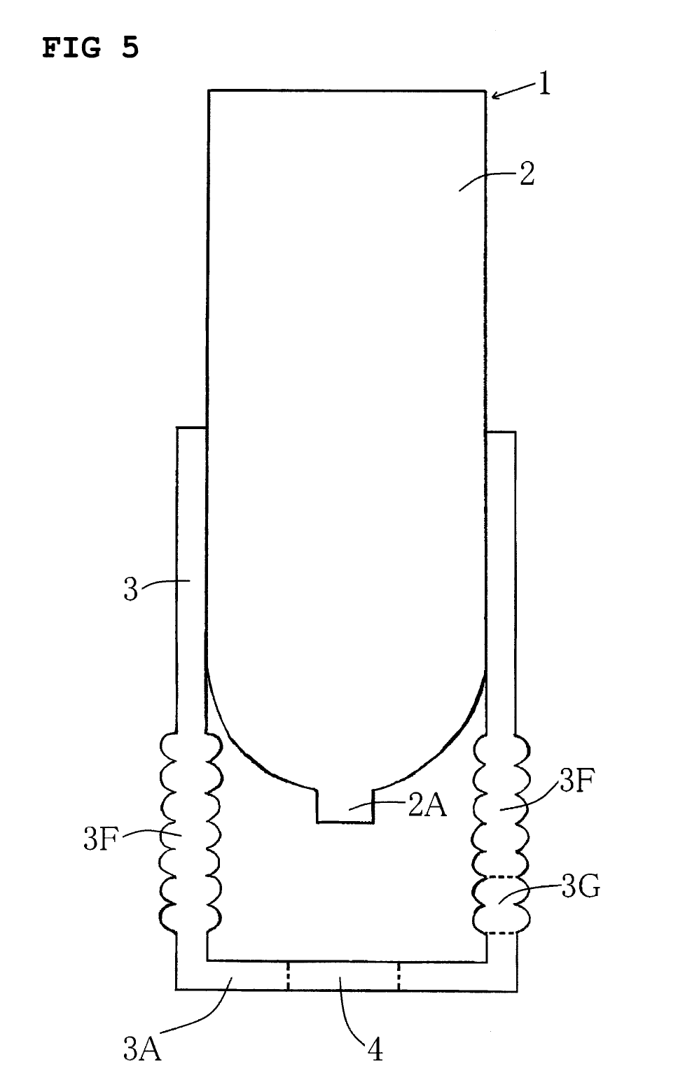

With respect also to contact means 3, there is no particular limitation. For example, contact means 3 may comprise an elastic pipe, as shown in FIG. 5. In FIG. 5, the rear end opening of pipe 3 as the contact means has inserted therein a forward region of adjuster means 2 (wherein the rear end portion of pipe 3 is attached to adjuster means 2), and the forward end opening of pipe 3 is targeting space 4. Circumferential pipe wall region 3F of the forward end opening of pipe 3 has therein finger insertion hole 3G which is capable of receiving the tip of palpation finger F of the user and causing the palpation fingertip to be positioned in targeting space 4. The attachment of the rear end portion of pipe 3 to adjuster means 2 can be made by attaching the inner wall of the rear end portion of pipe 3 to adjuster means 2, which can be performed by conventional methods, such as welding, fusion bonding, adhesion and screwing (e.g., attaching the inner wall of the rear end portion to adjuster means 2 by means of a screw; and screwing the inner wall of the rear end portion to adjuster means 2).

With respect to the elasticity of the pipe, there is no particular limitation so long as circumferential pipe wall region 3F of the forward end opening of the pipe has elasticity. As an example of shapes of circumferential pipe wall region 3F, there can be mentioned a shape of bellows.

With respect to the shape and size of the forward end opening (i.e., targeting space 4) of pipe 3, the explanation, given above, of the size of a through-hole applies, wherein the explanation is made in connection with the case where forward end portion 3A of contact means 3 comprises a plate having the through-hole.

With respect to the shape and size of hole 3G of circumferential pipe wall region 3F of the forward end opening of the pipe, there is no particular limitation so long as hole 3G is capable of receiving the tip of palpation finger F of the user and causing the palpation fingertip to be positioned in targeting space 4. Hole 3G may be a cut (slit) enabling finger F to be inserted to hole 3G.

If desired, the chiropractic apparatus of the present invention may further comprise a holding means by which the user may hold the chiropractic apparatus. With respect to the shape of the holding means, there is no particular limitation. For example, the holding means may be a member having a shape which can be held by hand. Alternatively, the holding means may be a member having a belt shape (belt member), which can be engaged with a hand or wrist of the user. With respect to the type of the belt member, there is no particular limitation. For example, the belt member may be a linear structure having the same configuration and use as a belt used as an apparel item. Alternatively, the belt member may be a ring structure, such as a rubber band. The holding means can be attached to at least one selected from the group consisting of adjuster means 2 and contact means 3. With respect to the materials used for producing the holding means, there is no particular limitation, and there can be used the same materials as used for producing adjuster means 2 and contact means 3, the materials being enumerated later. However, generally, it is preferred to use flexible materials, such as cloth, rubber, and flexible plastics.

Explained hereinbelow is the method for operating the chiropractic apparatus of the present invention (i.e., the method for performing a chiropractic treatment by using the chiropractic apparatus of the present invention).

The chiropractic apparatus of the present invention can be operated either by one hand or both hands of the user. Especially in the case where the forward end portion of the contact means comprises a plate, the chiropractic apparatus of the present invention can be easily operated even by one hand of the user. This is because, as shown in FIG. 4, when the plate which is the forward end portion of the contact means is placed on the body surface of the patient, the chiropractic apparatus can be held more stably even by one hand of the user.

Palpation can be performed either while keeping or while not keeping the forward end portion of the contact means positioned in contact with the palpation fingertip of the user. In the case where palpation is performed while keeping the forward end portion of the contact means positioned in contact with the palpation fingertip of the user, the time when the contact point of the body surface of the patient (i.e., the portion of the body surface of the patient corresponding to the target site for adjustment) has been determined by palpation, is simultaneous with the time when the targeting space at the forward end portion of the contact means is positioned at the contact point of the body surface of the patient as determined by palpation. On the other hand, in the case where palpation is performed while not keeping the forward end portion of the contact means positioned in contact with the palpation fingertip of the user, first, the contact point of the body surface of the patient has been determined by palpation, and then the forward end portion of the contact means is caused to be positioned in contact with the palpation fingertip of the user so as to cause the targeting space at the forward end portion of the contact means to be positioned at the contact point of the body surface of the patient at which the palpation fingertip is positioned. Important in the present invention is that the targeting space at the forward end portion of the contact means is caused to be positioned at the contact point of the body surface of the patient as determined by palpation, thereby enabling a correct adjustment.

For example, in the case where the forward end portion of the contact means comprises a rod as shown in FIG. 1, there may be used a palpation method in which palpation is performed by the palpation fingertip of one hand of the user while using the other, non-palpation hand of the user so as to keep the rod-shaped forward end portion of the contact means positioned in contact with the palpation fingertip of the user. Alternatively, there may be used a palpation method in which first, palpation is performed by the palpation fingertip of one hand of the user, and then the other, non-palpation hand of the user is used so as to cause the rod-shaped forward end portion of the contact means to be positioned in contact with the palpation fingertip of the user.

In the case where, as shown in any of FIGS. 2 to 5, the forward end portion of the contact means has a shape which can be easily engaged with the palpation fingertip of the user, the forward end portion of the contact means can be caused to be more correctly positioned in contact with the palpation fingertip of the user, thereby enabling a more correct adjustment. The methods for causing the palpation fingertip of the user to be engaged with the forward end portion of the contact means as in the cases of FIGS. 2 to 5, are as described below. In the case of FIG. 2, the forward end portion of the contact means comprises an arc, and the engagement is established by placing the palpation fingertip in the inside of the arc-shaped forward end portion of the contact means. In the case of FIG. 3, the forward end portion of the contact means comprises a loop, and the engagement is established by placing the palpation fingertip in the inside of the loop. In the case of FIG. 4, the forward end portion of the contact means comprises a plate having a through-hole therein, and the engagement is established by placing the palpation fingertip in the through-hole. In the case of FIG. 5, the contact means comprises a pipe which has an opening at a forward end thereof, and the engagement is established by placing the palpation fingertip in the opening at the forward end of the pipe.

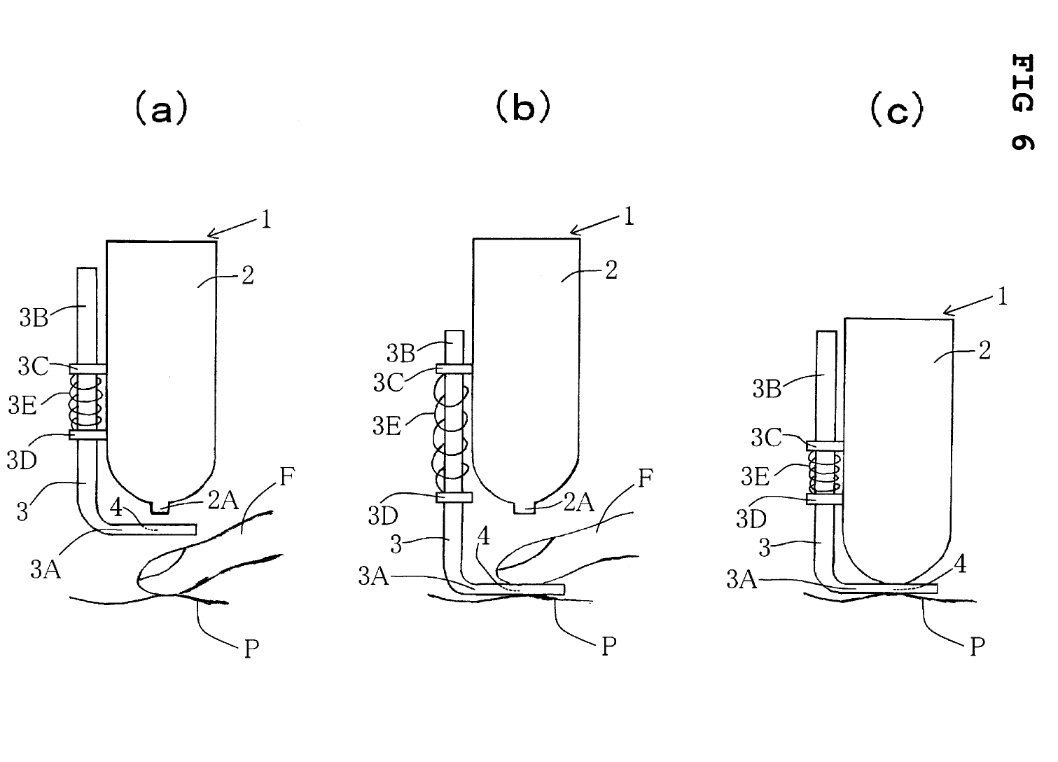

Hereinbelow, an example of a method for operating the chiropractic apparatus of the present invention is explained with reference to FIG. 6. FIG. 6 shows the case where palpation is performed while not keeping the palpation fingertip of the user positioned in contact with the forward end portion of the contact means.

As shown in (a) of FIG. 6, in the case of the use of the chiropractic apparatus of the present invention, first, body surface P of the patient is palpated with the tip of finger F of the user while pulling the skin of the portion of the body surface (and portions therearound) of the patient, expectedly corresponding to the target site for adjustment in the skeleton of the patient, so as to remove a skin slack, thereby determining the target site for adjustment. Finger F may be any of the thumb, index finger, middle finger, third finger and fourth finger. Finger F may be from either of the right and left hands.

Next, as shown in (b) of FIG. 6, forward end portion 3A of contact means 3 is moved to the tip of finger F of the user, the fingertip being positioned at the contact point of body surface P of the patient (i.e., the portion of body surface P of the patient corresponding to the target site for adjustment). When forward end portion 3A of contact means 3 has been moved to and positioned in contact with the tip of finger F of the user, targeting space 4 is positioned at the contact point of the body surface of the patient. Also, at this time, targeting space 4 is positioned right under forward end portion 2A of the chiropractic adjuster means, and there is a positional relationship such that, when forward end portion 2A of the adjuster means is moved to targeting space 4, forward end portion 2A of the adjuster means is placed in contact with the contact point of the body surface of the patient. If desired, a final palpation may be performed in the state as shown in (b) of FIG. 6 so as to confirm the correctness of the targeting of the target site for adjustment.

Subsequently, as shown in (c) of FIG. 6, forward end portion 2A of adjuster means 2 is moved to targeting space 4. Then, trigger member 2B of adjuster means 2 is pulled to bring adjuster means 2 into action. (In FIG. 2, trigger member 2B of adjuster means 2 is omitted.)

By performing the above-described operations, a very correct adjustment can be achieved. Thus, by performing the above-described operations, chiropractic treatment (adjustment) can be performed effectively and efficiently, as compared to the case of the use of conventional chiropractic techniques.

FIG. 6 shows a case where the longitudinal direction of adjuster means 2 is at right angles to body surface P of the patient; however, it is not necessary that the longitudinal direction of adjuster means 2 be at right angles to body surface P of the patient. The angle of the longitudinal direction of adjuster means 2 relative to body surface P of the patient may be an angle other than right angles, such as 45 degrees. The angle of the longitudinal direction of adjuster means 2 relative to body surface P of the patient may be chosen depending on the condition of the diseased site of the patient.

When forward end portion 3A of contact means 3 comprises a loop, it is preferred that palpation is performed in a state where the tip of finger F is engaged with the loop. When forward end portion 3A of contact means 3 comprises a plate having a through-hole therein, it is preferred that palpation is performed in a state where the tip of finger F is inserted in the through-hole. In the case where forward end portion 3A of contact means 3 comprises a plate having a through-hole therein, there are advantages in that a preload can be applied in the depthwise direction of the diseased site by pressing on the plate placed on the body surface of the patient, and also adjuster means 2 may be brought into action while applying a preload.

Chiropractic adjuster means 2 comprises:

a thrust member extending in a longitudinal direction of the chiropractic adjuster means and having a thrust head constituting a forward end portion of the chiropractic adjuster means,

a driving member for applying a longitudinal forward thrust to the thrust member, and

a trigger member for operating the driving member. In the present invention, the term “longitudinal forward thrust” that is applied to the thrust member, covers both of the case where the driving member moves forward the thrust member in the longitudinal direction thereof (i.e., the case where the thrust member is moved macroscopically), and the case where the driving member applies a mechanical impulse to the rear end of the thrust member, thereby generating a thrust in the form of a pressure wave propagating from the rear end to forward end of the thrust member in the longitudinal direction thereof (i.e., the case where the thrust member is not moved macroscopically, and only a pressure wave propagates from the rear end to forward end of the thrust member in the longitudinal direction thereof). Chiropractic adjuster means 2 used in the chiropractic apparatus of the present invention may have substantially the same basic structure, action mechanism and function as those of a conventional portable chiropractic apparatus, such as a so-called “activator”. Therefore, adjuster means 2 may be produced using the same design information and production materials as those of a conventional chiropractic apparatus which is disclosed, for example, in any of the above-mentioned Patent Documents 1 to 4.

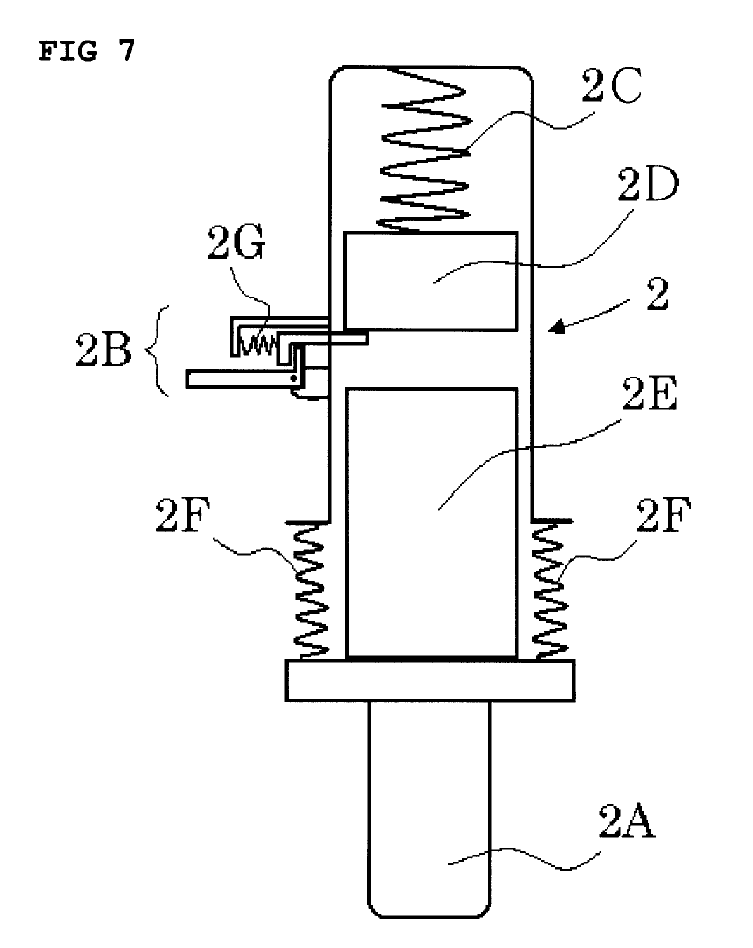

FIG. 7 is a diagrammatic view showing the internal structure of an example of chiropractic adjuster means 2. Adjuster means 2 shown in FIG. 7 comprises thrust head 2A, trigger member 2B, coil compression spring 2C, thrust driving block 2D, thrust rod 2E, and coil compression spring 2F. Trigger member 2B contains coil compression spring 2G. Coil compression spring 2C and thrust driving block 2D constitute the driving member for applying a longitudinal forward thrust to the thrust member. Thrust rod 2E and thrust head 2A are integral with each other so as to constitute the thrust member. Coil compression spring 2F serves as a cushion which can be compressed when forward end portion 2A of adjuster means 2 is pressed onto an object (such as the body surface of the patient). Thus, for example, even when adjuster means 2 as a whole is secured at a certain position, a downward movement of forward end portion 2A of adjuster means 2 can be achieved, because the thrust member (comprising thrust rod 2E and thrust head 2A) is capable of moving alone by virtue of the cushioning function of coil compression spring 2F. Adjuster means 2 as shown in FIG. 7 has a structure such that, by the driving member comprising coil compression spring 2C and thrust driving block 2D, the thrust member comprising thrust rod 2E and thrust head 2A is capable of moving forward (moving macroscopically) in the longitudinal direction thereof within the extension limit of coil compression spring 2F. Such structure is generally employed in the above-mentioned so-called “activator”.

By pulling trigger member 2B, adjuster means 2 is brought into action to thereby apply a thrust to the patient. As an example of a method for pulling trigger member 2B, there can be mentioned a method in which a trigger means (not shown) capable of being engaged with a finger of the user is connected with trigger member 2B through a wire, and trigger member 2B is pulled by moving the trigger means with the user's finger. The user's finger to be engaged with the trigger means may be the same as or different from finger F for palpation.

As another method for pulling trigger member 2B, there can be mentioned a method in which a wired or wireless communication means (not shown) is connected to trigger member 2B, and a switch (electronic trigger means) is turned on via the wired or wireless communication means to pull trigger member 2B. When this switch (electronic trigger means) is a foot switch device, the switching operation can be carried out with the user's foot.

With respect to the devices used for operating trigger member 2B of adjuster means 2 via the wired or wireless communication (i.e., wired or wireless communication means connected to trigger member 2B and a switch (electronic trigger means) to be used therewith), such devices can be easily produced based on the known electric/electronic engineering techniques applied in various fields or may be ones which are easily commercially available. As well known, due to the spreading of recent years, very small high-performance electro-communication devices or actuators for remote control or manipulation are widely applied as parts embedded in electric/electronic equipment of various fields. As an example of commercially available devices for wired remote control, there can be mentioned “TGA-mini” manufactured and sold by TOKI Corporation, Japan. Examples of available manufacturers capable of the services of designing and manufacturing devices for wired or wireless remote control include Kosugi Giken Co., Ltd., Japan, Osaka Jido Denki, Co., Ltd., Japan, and Itec Corporation, Japan. Thus, with respect to the electric/electronic devices or parts of relatively small sizes which are necessary for wired or wireless remote control of trigger member 2B used in the present invention, such devices or parts can be easily produced by applying the known technologies for wired or wireless remote control or manipulation, or such devices or parts may be easily commercially available, and, if desired, there are available manufacturers capable of the services of designing and producing such devices or parts.

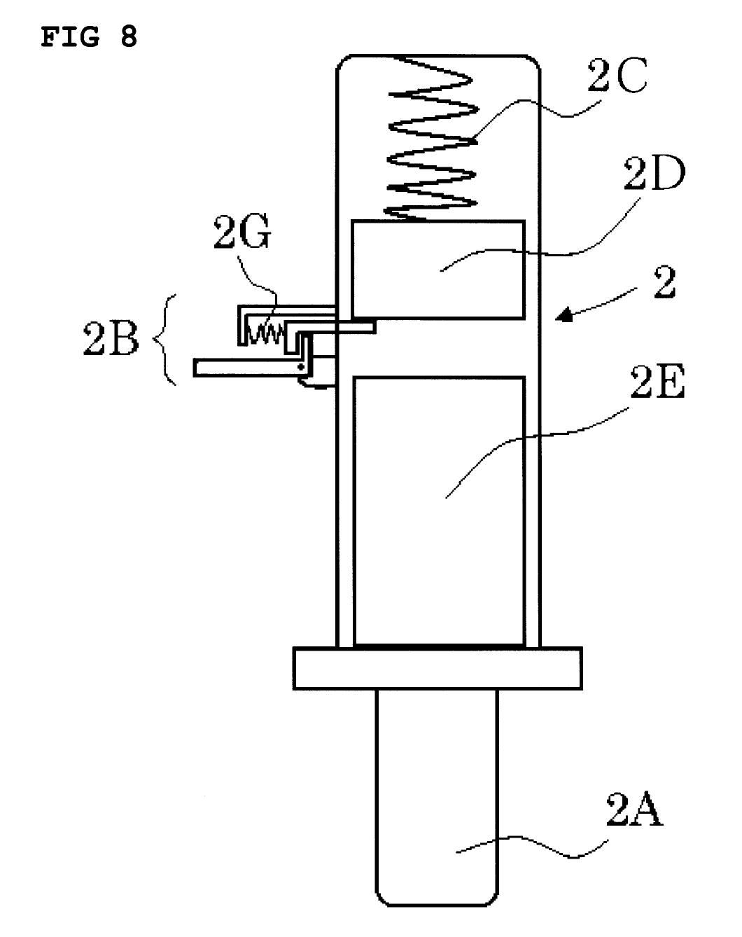

The diagrammatic view of the internal structure of another example of chiropractic adjuster means 2 is shown in FIG. 8. Adjuster means 2 shown in FIG. 8 has the same structure as that of adjuster means 2 shown in FIG. 7 except that adjuster means 2 shown in FIG. 8 does not have coil compression spring 2F shown in FIG. 7 and has a thrust member (comprising thrust rod 2E and thrust head 2A) fixed to the main body of adjuster means 2. Differing from adjuster means 2 shown in FIG. 7, adjuster means 2 shown in FIG. 8 does not give macro-scale movement of the thrust member independent of the main body of adjuster means 2 because the thrust member is fixed to the main body of adjuster means 2 as in the structure of a chiropractic apparatus used for the above-mentioned “Atlas Orthogonal Technique”. In the case of adjuster means 2 of FIG. 8 having such a structure, a driving member comprising coil compression spring 2C and thrust driving block 2D applies a mechanical impulse to the rear end of the thrust member so as to generate a thrust in the form of a pressure wave propagating from the rear end to forward end of the thrust member in the longitudinal direction of the thrust member, thereby applying a pressure (thrust) to the body surface of the patient. With respect to adjuster means 2 shown in FIG. 8, it is preferred that the thrust member is made of a metal (e.g., a steel) from the viewpoint of increasing the efficiency of propagation of the pressure wave through the thrust member.

The structure and action mechanism of adjuster means 2 are not limited to those shown in FIGS. 7 and 8. For example, as to the action mechanism, adjuster means 2 can be driven by any known mechanisms applied in the art for driving chiropractic adjusters, which use various driving forces, such as air or oil pressure, electromagnetic force and human power.

With respect to the materials used for producing the chiropractic apparatus of the present invention, there is no particular limitation so long as the object of the present invention can be achieved. It is preferred that the coil compression spring of adjuster means 2 is made of a metal (e.g., a steel). Other parts of adjuster means 2 may be made of, for example, plastics (such as a vinyl chloride resin, an acrylic resin, polypropylene and polycarbonate), steels including a stainless steel, and aluminum. The same as mentioned above applies to contact means 3.

When the forward end portion of contact means 3 comprises a plate, the lower surface of the plate is supposed to be contacted with the body surface of the patient during the palpation or the action of adjuster means 2. The lower surface of the plate (especially, at around the through-hole thereof) is preferably not so slippery against the body surface of the patient from the viewpoint of ease in removing a skin slack during the palpation and the viewpoint of prevention of the displacement of the through-hole of the plate from the contact point determined by the palpation. Therefore, specifically, it is preferred that the lower surface of the plate has many small bumps and dents formed at least at an area around the through-hole wherein, for example, bumps and dents each having a diameter of about 1 mm are formed such that the number of the bumps and dents is 10 or more per 1 cm² of the above-mentioned area around the through-hole. Alternatively, it is preferred that the above-mentioned area around the through-hole is roughened or at least this area of the plate is made of a rubber.

When contact means 3 comprises an elastic pipe, it is, as mentioned above, enough that contact means 3 is made of an elastic material with respect only to a circumferential pipe wall region around the forward end opening of the pipe as contact means 3. As an example of elastic materials, there can be mentioned a silicone rubber.

The palpation in a method for performing a chiropractic treatment by using the apparatus of the present invention can be carried out in the same manner as in the palpation in the conventional methods for performing a chiropractic treatment except for the following point. In a method for performing a chiropractic treatment by using the apparatus of the present invention, the palpation is carried out using the user's palpation fingertip which is placed in contact with the forward end portion of the contact means and which is received by the targeting space at the forward end portion of the contact means, to thereby cause the targeting space at the forward end portion of the contact means to be positioned at the portion of the body surface of the patient corresponding to the target site for adjustment. Alternatively, the portion of the body surface of the patient corresponding to the target site for adjustment may first be determined by palpation with the palpation fingertip of the user, whereafter the forward end portion of the contact means is moved so as to cause the targeting space at the forward end portion of the contact means to be positioned at the portion of the body surface of the patient (at which the palpation fingertip is positioned) corresponding to the target site for adjustment. For details of the palpation in the conventional methods for performing a chiropractic treatment, reference can be made to, for example, the above-mentioned Non-Patent Documents 1 to 3.

With respect to the bone to be targeted in the chiropractic treatment using the apparatus of the present invention, there is no particular limitation, and any bones which are generally considered as candidate targets in chiropractic treatments can be subjected to the chiropractic treatment using the apparatus of the present invention. Examples of bones which can be targeted in the chiropractic treatment using the apparatus of the present invention include the occipital bone (OC), the cervical vertebrae (C1 to C7), the thoracic vertebrae (Th1 to Th12), the lumbar vertebrae (L1 to L5), the pelvis, and joints of limbs.

Any chiropractors having ordinary experience and skill can easily carry out a chiropractic treatment using the apparatus of the present invention. Further, even chiropractors having no experience of using any of the conventional chiropractic apparatuses (adjusters) can easily use the apparatus of the present invention.

### INDUSTRIAL APPLICABILITY

By the use of the chiropractic apparatus of the present invention having a targeting function by tactile sensation, it becomes possible that a target site for chiropractic treatment (adjustment) is determined by palpation using a fingertip that is most sensitive in a human body, and then a thrust is correctly applied to the target site. Therefore, by the use of the chiropractic apparatus of the present invention, an adjustment can be performed more effectively and efficiently. Also, the chiropractic apparatus of the present invention is easily portable. Further, the chiropractic apparatus of the present invention is advantageous in that an adjustment can be performed even in a state where both hands of a user are kept in contact with the body of a patient, thereby enabling even a chiropractor not having a high level skill to perform easily a correct and effective adjustment of any bone of the entire body skeleton of the patient, such as the cranium, the spine, the lumbar vertebrae, the pelvis, and joints of limbs. With respect to the bone to be targeted in the chiropractic treatment using the chiropractic apparatus of the present invention, there is no particular limitation, and any bones which are generally considered as candidate targets in chiropractic treatments can be subjected to the chiropractic treatment using the apparatus of the present invention. Examples of bones which can be targeted in the chiropractic treatment using the apparatus of the present invention include the occipital bone (OC), the cervical vertebrae (C1 to C7), the thoracic vertebrae (Th1 to Th12), the lumbar vertebrae (L1 to L5), the pelvis, and joints of limbs.

I claim:

1. A chiropractic apparatus (1) comprising the following means (i) and (ii):
(i) a chiropractic adjuster means (2) comprising:
a thrust member (2E and 2A) extending in a longitudinal direction of said chiropractic adjuster means (2) and having a thrust head constituting a forward end portion (2A) of said chiropractic adjuster means (2),
a driving member (2C and 2D) for applying a longitudinal forward thrust to said thrust member (2E and 2A), and
a trigger member (2B) for operating said driving member (2C and 2D),
(ii) a contact means (3) attached to said chiropractic adjuster means (2), said contact means (3) being capable of relative movement with respect to said chiropractic adjuster means (2),
said contact means (3) having a forward end portion (3A) which is capable of contacting both the tip of a palpation finger (F) of a user and a body surface (P) of a patient,
said contact means (3) having, at said forward end portion (3A) thereof, a targeting space (4) which is capable of receiving the tip of the palpation finger (F) of the user and causing the palpation fingertip to be placed in contact with the body surface (P) of the patient,
said targeting space (4) being for receiving said forward end portion (2A) of said chiropractic adjuster means (2) and causing said forward end portion (2A) to be placed in contact with the body surface (P) of the patient,
wherein, by subjecting said contact means (3) and said chiropractic adjuster means (2) to relative movement with respect to each other, said forward end portion (2A) of said chiropractic adjuster means (2) is capable of moving to said targeting space (4) at the forward end portion (3A) of said contact means (3) so as to be placed in contact with the body surface (P) of the patient.

2. The chiropractic apparatus (1) according to claim 1, wherein said forward end portion (3A) of said contact means (3) comprises an arc, and the inside of the arc is said targeting space (4).

3. The chiropractic apparatus (1) according to claim 1, wherein said forward end portion (3A) of said contact means (3) comprises a loop, and the inside of the loop is said targeting space (4).

4. The chiropractic apparatus (1) according to claim 1, wherein said forward end portion (3A) of said contact means (3) comprises a plate, said plate having upper and lower surfaces and a through-hole extending between said upper and lower surfaces, wherein said through-hole is said targeting space (4),
wherein said chiropractic adjuster means (2) is positioned on the side of the upper surface of said plate, and the lower surface of said plate is capable of being placed in contact with the body surface (P) of the patient.

5. The chiropractic apparatus (1) according to claim 1, wherein said contact means (3) comprises an elastic pipe having rear and forward end openings,
said rear end opening of said elastic pipe having inserted therein a forward region of said chiropractic adjuster means (2),
said forward end opening of said elastic pipe being said targeting space (4), and
wherein said forward end opening of said elastic pipe has a circumferential pipe wall region (3F) having therein a finger insertion hole (3G) which is capable of receiving the tip of the palpation finger (F) of the user and causing the palpation fingertip to be positioned in said targeting space (4).
